(19) United States
(12) Patent Application Publication
(10) Pub. No.: US 2007/0287687 A1
(43) Pub. Date: Dec. 13, 2007

(54) Method for removing debris from tooth and bone surfaces using irrigation solution

(76) Inventors: Carolyn M. Primus, Bradenton, FL (US); Kenneth S. Peterson, Lancaster, PA (US); Mahmoud Torabinejad, Loma Linda, CA (US)

Correspondence Address: DENTSPLY INTERNATIONAL INC, 570 WEST COLLEGE AVENUE, YORK, PA, 17404, US

(21) Appl. No.: 11/650,157

(22) Filed: Jan. 5, 2007

Related U.S. Application Data
(63) Continuation of application No. 11/451,839, filed on Jun. 13, 2006.

(51) Int. Cl. A61K 31/65

(52) U.S. Cl. 514/152; 433/216

(57) ABSTRACT

Methods for removing debris and bacteria from tooth and orthopedic surfaces using irrigation solutions are provided. The solution includes disinfectant, surfactant, and organic acid components. Disinfectants such as tetracycline and doxycycline compounds can be used. Suitable surfactants include cocamidopropyl betaine compounds and polyoxypropylene-polyoxyethylene copolymers. The solution is particularly suitable for removing smear layers from tooth surfaces during root canal treatment and other dental procedures such as restoration, reconstruction, and periodontal work. The solutions also can be used to remove smear layers from bone surfaces.

### CROSS-REFERENCE TO RELATED APPLICATIONS

This application is a continuation of U.S. patent application Ser. No. 11/451,839 having a filing date of Jun. 13, 2006, the entire contents of which are hereby incorporated by reference.

### BACKGROUND OF THE INVENTION

1. Field of the Invention

The present invention relates to methods for removing undesirable substances from tooth surfaces during dental procedures. The methods remove buildup of debris and bacteria formed during preparation of tooth surfaces in procedures such as root canal treatment, restoration, dental reconstruction, periodontal procedures, and the like. The methods also are suitable for preparing bone for reconstruction or restoration.

2. Brief Description of the Related Art

As a consequence of pathological changes in the dental pulp, the root canal system acquires the capacity to harbor many species of bacteria, their toxins and their by-products. The microorganisms present in infected root canals are predominantly gram-negative anaerobes that are seeded into the root canals from direct pulp exposures (caries or traumatic injuries) or coronal microleakage. The morphology of root canals is very complex and mechanically prepared root canals contain areas that cannot be reached by endodontic instruments. The microorganisms present in the root canal not only invade the anatomic irregularities of the root canal system, but also invade the dentinal tubules.

In the root, dentinal tubules extend from the intermediate dentin just inside the cementum-dentin junction to the pulp-predentin junction. Tubules are approximately 1 μm in diameter near the cementum-dentin junction and approximately 2.5 μm near the pulp-predentin junction. The number of dentinal tubules per square millimeter varies from 8,000 to 57,000. At the periphery of the root at the cemento-enamel junction, the number has been estimated to be approximately 15,000 per square millimeter.

Many studies have shown that currently used methods of cleaning and shaping produce a smear layer that covers root canal walls. The smear layer is produced as a result of instrumentation and its content is forced into the dentinal tubules to varying distances. Moodnik, R. M., Dorn, S. O., Feldman, M. J., Levey, M., and Borden, B. G., J. Endodon., 1976, 2, 261-266; Cengiz, T., Aktener, B. O., and Piskin, B., Int'l. Endodon. J., 1990, 23, 163-171. Cengiz, et al. suggested that the penetration of smear material into the dentinal tubules is probably caused by capillary action generated between the dentinal tubules and the smear material.

In 1975, McComb and Smith described the smear layer in endodontics. McComb, D., and Smith, D. C., J. Endodon., 1975, 1, 238-242. It was later characterized as consisting of a superficial layer on the surface of the canal wall that averages between 1-2 μm in thickness, and a deeper layer packed into the dentinal tubules to a depth of up to 40 μm. Cameron, J. A., J. Endodon., 1983, 9, 289-292; Mader, C. L., Baumgartner, J. C., and Peters, D. D., J. Endodon., 1984, 10, 477-483. The smear layer consists of organic and inorganic substances that include fragments of odontoblastic processes, microorganisms and necrotic materials. A number of studies have shown that presence of smear layer can prevent penetration of root canal medications and sealers into the dental tubules. In addition, they have shown that removal of the smear layer results in better adaptation between root canal filling materials and the dentinal walls.

Smear layers are also formed when tooth material is removed preparatory to restoration or other dental work, as it is for root canal situations. Moreover, in the restoration of bone, such as in orthopedic restorations, debris layers similar in many respects to endodontic smear layers are also formed.

In recent years, professionals in the dental and medical field have attempted to develop methods for removing smear layers from tooth and bone surfaces. One effective method involves treating the prepared tooth and bone surfaces with a solution of disinfectant, surfactant, and organic acid as described in Torabinejad et al., United States Patent Application Publication Nos. US 2003/0138383 and US 2003/0235804 (“Torabinejad published applications”). As described in the Torabinejad published applications, the disinfectant used to make the solution is preferably a member of the tetracycline family, which includes tetracycline-HCL, minocycline, and doxycycline; the surfactant is preferably selected from the group of sorbitan esters or polysorbates; and the acid is preferably an organic acid, for example, citric acid. Such formulations are generally effective for removing the smear layer and sterilizing endodontic root surfaces. The formulations also may be used to remove smear layers from orthopedic and bone restoration sites within or without the oral cavity. One disadvantage, however, with the disinfectant/surfactant/acid solutions described in the Torabinejad published applications is that their appearance may become unstable over time. The solutions may become cloudy and discolored. Also, precipitates and globules may form in the solution. Although the solution remains completely effective for its intended purpose, dental and medical professionals may have concerns over its appearance. In some cases, dentists, endodontists, and others may be hesitant to use such cloudy solutions when treating patients. Accordingly, there is a need for improved disinfectant solutions having higher stability and shelf life over solutions used in the past. Such solutions should be esthetically pleasing and remain effective in breaking down smear layers on prepared tooth and bone surfaces. The present invention provides such solutions and methods for using such solutions.

### SUMMARY OF THE INVENTION

The present invention provides a method for irrigating prepared tooth and bone surfaces to remove smear layers. A solution comprising disinfectant, surfactant, and organic acid is used in the method. The surfactant is selected from the group consisting of cocamidopropyl betaine compounds and copolymers of polyoxypropylene-polyoxyethylene. Preferably, the disinfectant is tetracycline or doxycycline, and the acid is citric acid. The solution of this invention can be made from two parts, wherein Part A is a liquid carrier comprising water, surfactant, and organic acid and Part B is a disinfectant powder, preferably doxycycline powder. The liquid carrier has relatively high stability and long shelf life. The liquid carrier remains generally clear over an extended period of time providing an improved esthetic appearance.

Such solutions are useful in a variety of dental treatment applications, including, but not limited to, root canal therapy; preparation of cavities; cosmetic and reconstructive dentistry such as caps, crowns, bridges, veneers, and the like; other endodontic procedures; and periodontic procedures. Such solutions also are useful for preparing bone for reconstruction or restoration.

### DETAILED DESCRIPTION OF THE PREFERRED EMBODIMENTS

The term “smear layer” as used herein, is well known to persons skilled in the art of dentistry and refers to the complex accumulation of organic and inorganic debris resulting from the mechanical preparation of a tooth surface. The smear layer comprises cutting debris, tooth particles, microorganisms, necrotic material, and other substances resulting from preparation, and typically includes a superficial layer on the surface of a prepared tooth along with a layer or layers that are packed into the adjacent dentinal tubules at varying depths up to about 40 μm. In the context of orthopedics, “smear layer” refers to similar layers in prepared bone sites.

The term “disinfectant”, as used herein, refers collectively to compositions that are able to suppress or eliminate bacterial or other microorganisms found in endodontic or periodontic sites. The term “disinfectant” includes antibiotics and antimicrobials as these terms are understood in pharmaceutical science.

The solution of this invention comprises disinfectant, surfactant, and acid. In a preferred embodiment, the disinfectant is an antibiotic. It will be apparent to one skilled in the art that the antibiotic should be stable in the acidic solutions of which it forms a part, should be compatible with the other components of the solution, and should retain its effectiveness for at least the time of preparation of the solution and its application and residence time on or in the prepared tooth or bone surface. Examples of such antibiotics include, but are not limited to, ansamycins, including rifamycins; cephalosporin; macrolides such as clarithromycin, josamycin, and oleandomycin; most polypeptides, such as bacitracin, capreomycin, enduracidin, enviomycin, gramicidin, mikamycin, ristocetin, thiostrepton, tyrocidine, viomycin, and virginiamycin; all tetracycline compounds, such as apicycline, chlortetracycline, clomocycline, demeclocycline, doxycycline, guamecycline, lymecycline, mecleocycline, methacycline, minocycline, oxytetracycline, penimepicycline, pipacycline, rolitetracycline, sancycline, mupirocin, and tetracycline-HCl; and tuberin. Most quinolones such as ciprofloxacin, gatifloxacin, and moxifloxacin are not preferred, as they are weak bases and have decreased effect in acidic solutions. Additionally, most B-lactam antibiotics, particularly penicillins, are also not preferred, as they are generally unstable in acidic solutions. Exceptions, however, are amoxycillin, an acid-stable member of the penicillin family, and similar compounds.

Tetracyclines are broad-spectrum antibiotics that are effective against a wide range of microorganisms. They include tetracycline-HCl, minocycline, and doxycycline. Tetracyclines are bacteriostatic in nature and are generally more effective against gram-positive bacteria compared to gram-negative bacteria. A reference to tetracycline shall be taken to include all members of the tetracycline family. A number of studies have shown that tetracyclines significantly enhance healing after surgical periodontic therapy. Members of the family of tetracyclines are preferred for use herein. Tetracyclines are preferred for a number of reasons. One reason they are preferred is because they have many unique properties along with their antimicrobial effect. For example, tetracycline-HCl has a low pH in concentrated solution and thus can act as a calcium chelator, and cause enamel and root surface demineralization. Tetracycline-HCl's surface demineralization of dentin is comparable to that seen using citric acid. In addition, it has been shown that tetracycline-HCl is a sustentative medication and becomes absorbed and released from tooth structures such as dentin and cementum. The use of tetracycline is also preferred because a very low portion of the general population exhibits allergies or other sensitivities to tetracycline. Doxycycline, a broad-spectrum antibiotic synthetically derived from oxytetracycline, is particularly preferred. Doxycycline is available as doxycycline monohydrate; doxycycline hyclate; doxycycline hydrochloride hemiethanolate hemihydrate; and doxycycline calcium for oral administration.

Antimicrobial compounds may be used in accordance with this invention. It will be apparent to one skilled in the art that the antimicrobial compound should be stable in the acidic solutions of which it forms a part, should be compatible with the other components of the solution, and should retain its effectiveness for at least the time of preparation of the solution and its application and residence time on or in the prepared tooth or bone surface. Examples of such antimicrobial compounds include, but are not limited to, chlorhexidine compounds. Chlorhexidine gluconate is preferred. One example of a suitable chlorhexidine gluconate solution is a commercially available 0.12% solution known as “Peridex™”. Additionally, the use of chlorhexidine gluconate has been found to be especially desirable in patients who exhibit sensitivity or allergy to tetracycline compounds.

In the improved solution of this invention, the surfactant (detergent) is selected from the group consisting of cocamidopropyl betaine compounds and copolymers of polyoxypropylene-polyoxyethylene. Cocamidopropyl betaine compounds are amphoteric surfactants and are available from Lonza, Inc. (Allendale, N.J.) under the tradenames, Lonzaine C® and CO®. These cocamidopropyl betaine compounds are mild, high foaming, biodegradable surface active agents. Mixtures comprising Lonzaine C® and CO® surfactants and other surfactants may be prepared, since the Lonzaine C® and CO® surfactants are highly compatible with anionic, cationic, and non-ionic surfactants. Polyoxypropylene-polyoxyethylene copolymers are non-ionic surfactants and are available from BASF (Mount Olive, N.J.) under the tradename, Pluronic®. Such polyoxypropylene-polyoxyethylene copolymers may be referred to as poloxamer block copolymers. It has been discovered that the cocamidopropyl betaine surfactants and polyoxypropylene-polyoxyethylene copolymer surfactants are stable in the acidic solution for long periods and at elevated temperatures. The cocamidopropyl betaine surfactants and polyoxypropylene-polyoxyethylene copolymer surfactants help improve the shelf-life and stability of the acidic solution. In addition, the surfactants help to enhance the wetting effect of the solution so that it can better break-down the smear layer on the surface of the tooth or other surface.

It will be apparent to one of skill in the art of dentistry that the acid used in the solution should be suitable for dental applications. Thus, the acid should be nontoxic in the applicable concentration and amount used in the irrigation process and should also be compatible with the surfactant and disinfectant selected as the other components of the solution. Preferred acids must also be capable of dissolving the organic and inorganic components of the smear layer within the chosen exposure time, but without inducing unwanted erosion of the tooth and surrounding surfaces.

In another preferred embodiment, the acid is an organic acid, preferably having pKa values between 1.5 and 5. Further preferred are carboxylic acids or other acids with a polar nature and pKa values between 2 and 5. In a further preferred mode of the present invention, an acid with a pKa value between about 2.75 and 3.75 is used. One exemplary member of the preferred class is citric acid. Citric acid is particularly suitable when tetracycline is chosen as the disinfectant, because citric acid does not diminish or otherwise alter the antibacterial effect of tetracycline.

It will be apparent to one skilled in the art, however, that stronger acids may also be preferred for use in the present invention provided that the time of application of the solution is shortened accordingly. As such, stronger acids including, but not limited to, chloracetic, maleic, saccharic, tartaric, and polyacrylic may be used, having pKa values ranging from about 0.5 to about 3.0. Mixtures may also be used. In some embodiments inorganic acid, specifically phosphoric acid may find utility so long as the essential properties of the solution are maintained.

The disinfectants are present in the solutions of the present invention in weight percentages of from about 1 to about 5 percent of the solution and preferably in amounts of from about 2 to about 4 weight percent, with amounts of about 3 percent being even more preferred, especially when the disinfectant is a tetracycline.

The surfactant is preferably present in the solutions of the invention in weight percentages of from about 0.1 to about 1.5 percent of the solution, with amounts of from about 0.25 to about 1.0 percent being more preferred. Amounts by weight of about 0.5 percent are generally most preferred depending upon the surfactant, especially when the surfactant is a cocamidopropyl betaine compound or copolymer of polyoxypropylene-polyoxyethylene.

The acids of the invention are present in the solutions in amounts of from about 0.5 to about 10 percent by weight of the solution, preferably from about 3 to about 6 percent. More preferred are solutions having weight percentages of acid, especially organic acid, of from about 4 to about 5 percent. The pH value of the solutions of this invention will vary based upon the strength of the organic acid component used and concentration of the acid in the solution. However, in general, the solutions have a pH value of no greater than about 4 and preferably within the range of about 1 to about 4.

In general, the solutions of the invention are aqueous and water comprises the bulk of the balance of the composition. Solutions of the invention may also include other compounds, however, so long as they do not interfere with the essential functions of the principal components, do not cause them to degrade and do not interfere with the convenience and utility thereof. Such additional additives may include fluorescing agents, colorants, flavorants, stabilizers, and other materials conventionally added to dental or orthopedic solutions. One particularly useful adjuvant may be chelating agents capable of rendering chelatable materials, especially metals, soluble. Indeed, use of a polyfunctional acid may achieve this goal. It will be recognized by one of skill in the art that regardless of the components or additives in the solution, the resulting solution should be sterile so that the objectives of the invention are achieved. In all cases, such materials are present in effective amounts to accomplish their objectives.

The present invention is directed to methods for sterilizing and removing the smear layer on a prepared tooth or canal surface comprising irrigating the surface with a solution comprising disinfectant, surfactant, and acid. In preferred modes of the invention, the disinfectant is an antibiotic that is sufficiently stable in an acidic environment. It is further preferred that the antibiotic be a tetracycline compound. More preferably, the tetracycline compound is doxycycline, particularly in the form of doxycycline hyclate.

In another preferred aspect of the present invention, the acid is an organic acid, preferably having a pKa between 1.5 and 5. In a further preferred embodiment, the organic acid has a pKa between 2 and 4; preferably between 2.75 and 3.75, such as that of citric acid. In a further embodiment, the acid is phosphoric acid.

In one embodiment, the antibacterial cleanser solution of this invention is made from two parts. Part A of the product is a liquid carrier comprising water, surfactant, and organic acid—this liquid part can be stored in a luer-lock syringe. Part B is a disinfectant powder, preferably doxycycline powder—the powder part can be stored in a medication bottle with an opening adapted for receiving the syringe. In practice, a dentist inserts the luer-lock syringe containing the liquid carrier into the bottle and locks the syringe in place by twisting it. Once the syringe is locked in place, the dentist depresses the syringe plunger to fill the bottle with the liquid carrier. The inter-locked syringe and bottle assembly can be gently rocked back and forth allowing the liquid to fully dissolve the tetracycline powder. Then, the dentist draws the solution from the bottle and into the syringe. The dentist is now ready to express the cleanser solution into the root canal of the tooth being treated. First, the dentist places an irrigation needle/probe onto the syringe. Then, the dentist slowly expresses some of the solution into the root canal of the tooth. An endodontic file can be used to mechanically agitate the solution. The solution is allowed to remain in the canal of a sufficient period of time so that it can soak into the root canal surfaces. During this step, the solution cleans and disinfects the canal. The remaining solution in the syringe can be used to rinse and flush the canal.

It should be understood that the above-described starting materials (an aqueous liquid part comprising surfactant and organic acid and a powder part comprising disinfectant) are only one example of the materials that can be used to prepare the antibacterial, cleanser solution of this invention. The foregoing embodiment is meant for illustration purposes and should not be considered restrictive. Workers skilled in the art will appreciate that other materials and methods can be used to make the solutions of this invention. For instance, a powder part comprising disinfectant and surfactant may be prepared and combined with a liquid part comprising an organic acid component. In another embodiment, the powder part may include the disinfectant and organic acid, while the liquid part includes the surfactant. These are only some examples of materials and methods that can be used without departing from the spirit and scope of the present invention.

The methods of the present invention can be used on surfaces of instrumented root canals, sites prepared for periodontal procedures, sites prepared for tooth restoration or reconstruction, and sites prepared for bone restoration or reconstruction. In a preferred mode of the present invention, the prepared tooth surface is irrigated for between 1 minute and 1 hour, preferably between 1 and 30 minutes and more preferably from about 1 to about 10 minutes.

Although the methods of using the solution described above are exemplary for the present invention, there are other embodiments that may be foreseen by those skilled in the art. The solution of the present invention can also have use in preparation for implants in the animal body. Such foreseeable preparations include use with cochlear, cranial, sternum, other custom implants or functional shapes made for the body. Other embodiments can be used for preparation for insertion of universal plates for orthopedic use, bone screws, rods & pins for orthopedic use (IM nails, femoral rods or plugs, long bone fractures, etc.), tendon anchors, suture anchors and tacks, graft retainers and marrow sampling ports.

For use in connection with removal of smear layer from bony preparations, either in the mouth or upon skeletal bone, a prepared site is irrigated for from 1 minute to one hour, preferably from 1 minute to about 30 minutes, with from about 1 to 10 minutes being preferred. By “irrigation” is meant contacting the site with the solution. It is preferred to provide a flow of such solution over the surfaces of the site, however, this need not be performed continuously. Flow of solution may be accompanied by air entrainment to assist in smear layer removal through action of the ensuing bubbles. Other physical means of assisting with smear layer removal may accompany irrigation and all such are encompassed hereby. Following irrigation, the site is dried and used for the intended restoration.

It has further been discovered that the solutions of the present invention can be particularly effective when used following an initial rinse comprising NaOCl. In such applications, NaOCl may be used as an irrigant in conjunction with instrumentation of a surface. Following instrumentation and use of the NaOCl rinse, a final rinse of disinfectant/surfactant/acid solution may be applied to the surface. This method is believed to have an improved effect on smear layer removal because the initial NaOCl rinse removes some organic materials from the smear layer, while the disinfectant/surfactant/acid solution removes inorganic and residual organic materials. When NaOCl is used in conjunction with the solution of the present invention, it is preferred that the concentration of NaOCl be between about 1% and about 6% by weight, and the concentration is most preferably between about 1.3% and about 5.25% by weight. Because testing indicates that there is no significant difference in performance within this concentration range, and high concentrations of NaOCl are known to be more toxic than lower concentrations, it is recommended that NaOCl at the lower end of the given concentration range be used. A method comprising the use of an initial rinse of a solution of 1.3% NaOCl by weight followed by a final rinse of a solution comprising doxycycline, polysorbate 80, and citric acid is particularly desirable.

The shelf life of the antibacterial, cleanser solution is improved by using surfactants selected from the group consisting of cocamidopropyl betaine compounds and polyoxypropylene-polyoxyethylene copolymers. As discussed above, the solution of this invention can be made from two parts, wherein Part A is a liquid carrier comprising water, surfactant, and organic acid and Part B is a disinfectant powder, preferably doxycycline powder. One problem with some surfactants is that they may react with the organic acid component in the liquid carrier and form a white, hazy precipitate over time. The liquid carrier, which is customarily stored in a luer-lock syringe, can become cloudy and develop an esthetically non-pleasing appearance over time. One factor that determines the rate of clouding in the liquid carrier is the temperature at which the liquid is stored. For example, if the liquid carrier is stored at room temperature, the liquid may start to cloud at about six months. On the other hand, if the liquid carrier is stored at 60° C., the liquid may start to cloud within a week. When such liquid carriers are mixed with the disinfectant powder, the resulting solution also develops a cloudy, hazy appearance. Such solutions are still effective in removing smear layers from tooth and bone surfaces; however, the solutions have an esthetically non-pleasing appearance. Now, it has been found that surfactants comprising cocamidopropyl betaine compounds and polyoxypropylene-polyoxyethylene copolymers are particularly stable in liquid carriers containing organic acid. It is believed that these surfactants do not react strongly with the acid component. As a result, the liquid carrier is more stable, and it does not tend to form a precipitate. The liquid has a more esthetically pleasing appearance. The liquid can be mixed with the disinfectant powder to form an antibacterial cleanser solution suitable for removing smear layers from tooth and bone surfaces. The surfactant is preferably present in the solution in an amount within the range of about 0.05 to about 5 weight percent and more preferably in the range of 0.05 to 1.5 weight percent. Improving the shelf-life and stability of the liquid carrier means that the shelf-life and stability of the ultimate antibacterial cleanser solution also is improved.

The invention is further illustrated by the following examples, which are not intended to be limiting.

### EXAMPLES

### Example 1

Removal of Smear Layer from Root Canal Walls.

Extracted maxillary and mandibular human teeth were used for this study. Twenty-eight extracted maxillary and mandibular single-rooted human teeth were used. Mandibular incisors and teeth with previous root canal treatment were excluded. The teeth were randomly divided into three (3) experimental groups of eight (8) teeth each and a control group of four (4) teeth. The groups were organized according to the type of irrigation solutions and final rinses used during and after instrumentation as described further below.

Each canal was instrumented by using a combination of passive step-back and rotary 0.06 taper nickel-titanium files. After removing the crown of each tooth, a K-type file (size 10 or 15) was used to determine the working length by penetrating the apical foramen and pulling back into the clinical apical foramen. The apical foramen of each tooth was enlarged to a size 35 K-file. NaOCl solution 1.3% was used as intracanal irrigation solution during instrumentation. One milliliter of irrigation solution was used to irrigate the root canal between each hand and rotary instrument. A total of 10 ml of irrigation solution was used in each root canal. The irrigation solution was delivered with a 23-gauge monoject endodontic needle (Kendall, Tyco/Healthcare), which penetrates to within 1 to 2 mm from the working length in each canal. Each canal was filled with an irrigation solution during instrumentation as described further below. The instrumentation time for each root canal was 15 to 20 min.

The canals were treated with 5 ml of one of the following solutions to compare the control and experimental solutions as a final rinse on the surface of instrumented root canals.

1. Sterile distilled water (positive control).

2. BioPure™ MTAD™ antibacterial root canal cleanser comprising doxycycline, polysorbate 80 surfactant, citric acid, and water, available from Dentsply Tulsa (Tulsa, Okla.).

3. Solution of the present invention comprising doxycycline, Lonzaine CO® surfactant (cocamidopropyl betaine compounds), citric acid, and water (3% doxycycline solution).

After instrumentation, the canals of the teeth in Group 1 were irrigated with the solution of Group 1; the canals of the teeth in Group 2 were irrigated with the solution of Group 2; and the canals of the teeth in Group 3 were irrigated with the solution of Group 3. Each canal was initially irrigated with 1 ml of one of the above solutions. After 5 min, each canal in Group 2 was irrigated with 4 ml of the solution in Group 2, and each canal in Group 3 was irrigated with 4 ml of the solution in Group 3 as a final rinse. The irrigation solutions were delivered with a 23 gauge monoject endodontic needle (Kendall, Tyco/Healthcare), which penetrates to within 1 to 2 mm from the working length in each canal. The total exposure time for the final rinse was approximately 1 min. Then the canals were irrigated with 10 ml of sterile distilled water and dried with paper points.

The teeth were prepared for examination using a SEM as follows. The teeth were split longitudinally (using diamond disc and chisel), and half of each tooth was placed in a 2% glutaraldehyde solution for 24 h. The other half of each tooth was discarded. The fixed specimens were rinsed three times with a sodium cacodylate buffered solution (0.1 M, pH 7.2), then incubated in osmium tetroxide for 1 hour, then were rinsed again twice with a sodium cacodylate buffered solution (0.1 M, pH 7.2). The dehydration process was performed by placing specimens in ascending concentrations of ethyl alcohol (30-100%), and storing them in a desecrator for at least 24 h. Each specimen was mounted on an aluminum stub and coated with 25 μm of gold-palladium and examined under a scanning electron microscope.

The specimens were coded and examined in a blind manner. Three investigators scored the presence or absence of smear layer on the surface of the root canal or in the dentinal tubules at the coronal, middle, and apical portion of each canal according to the following criteria:
1=No smear layer. No smear layer on the surface of the root canals; all tubules were clean and open.
2=Moderate smear layer. No smear layer on the surface of the root canal, but tubules contained debris.
3=Heavy smear layer. Smear layer covered the root canal surface and the tubules.

The same investigators scored the degree of erosion of dentinal tubules as follows:
1=No erosion. All tubules looked normal in appearance and size.
2=Moderate erosion. The peritubular dentin was eroded.
3=Severe erosion. The intertubular dentin was destroyed and tubules were connected to each other.

Results

The Cochran-Mantel-Haenszel method was used to analyze the data. The ratings for each tooth is given in Tables A-1 to A-4 for erosion and smear layer removal, in each of 3 areas of the root canal.

TABLE A-1
Smear Layer & Erosion of Dentin by
Group 1, sterile distilled water

| | Smear layer | | | Dentinal erosion | | |
|---|---|---|---|---|---|---|
| Sample no. | Apical | Middle | Coronal | Apical | Middle | Coronal |
| #1 | 3 | 3 | 3 | — | — | — |
| #2 | 3 | 3 | 3 | — | — | — |
| #3 | 3 | 3 | 3 | — | — | — |
| #4 | 3 | 3 | 3 | — | — | — |

TABLE A-2
Smear Layer & Erosion of Dentin by Group
2, solution with Polysorbate(Tween)80

| | Smear layer | | | Dentinal erosion | | |
|---|---|---|---|---|---|---|
| Sample no. | Apical | Middle | Coronal | Apical | Middle | Coronal |
| #1 | 3 | 1 | 1 | 1 | 1 | 1 |
| #2 | 3 | 2 | 1 | 1 | 1 | 1 |
| #3 | 1 | 1 | 2 | 1 | 1 | 1 |
| #4 | 2 | 2 | 2 | 1 | 1 | 1 |
| #5 | 2 | 1 | 1 | 1 | 1 | 1 |
| #6 | 3 | 1 | 1 | 1 | 1 | 1 |
| #7 | 2 | 2 | 1 | 1 | 1 | 1 |
| #8 | 3 | 1 | 1 | 1 | 1 | 1 |

TABLE A-3
Smear Layer & Erosion of Dentin by
Group 3, solution with LONZAINE CO

| | Smear layer | | | Dentinal erosion | | |
|---|---|---|---|---|---|---|
| Sample no. | Apical | Middle | Coronal | Apical | Middle | Coronal |
| #1 | 1 | 1 | 1 | 1 | 1 | 1 |
| #2 | 1 | 1 | 1 | 1 | 1 | 1 |
| #3 | 2 | 2 | 1 | 1 | 1 | 1 |
| #4 | 3 | 1 | 1 | 1 | 1 | 1 |
| #5 | 2 | 1 | 1 | 1 | 1 | 1 |
| #6 | 3 | 2 | 2 | 1 | 1 | 1 |
| #7 | 1 | 1 | 2 | 1 | 1 | 1 |
| #8 | 2 | 1 | 1 | 1 | 2 | 1 |

There was no statistical difference between the Tween 80 surfactant (Group 2) and the Lonzaine CO surfactant (Group 3)—containing solutions with regard to smear layer removal in any area of the root canals: apical, middle, or coronal. The solution of Group 3 handled as well as the solution of Group 2 and did not produce any more bubbles upon gentle mixing.

### Example 2

Effect of Surfactants on Stability of Solutions

In the following example, the stability of solutions containing an acid component and different surfactants (surfactants) was evaluated. Solutions of 4.4% citric acid and 0.5% surfactant were prepared. The solutions were placed in clear glass bottles, and the bottles were placed on a hot-plate to approximate accelerated aging conditions. The temperature of the liquids in the bottle was about 60° C., although some variations occurred depending upon the position of the bottle. The solutions were observed at different times and the results are reported below in Table 1.

TABLE 1

| Sam- | | |
|---|---|---|
| ple | Surfactant | Observations |
| 1 | TRITON X-200 | Cloudy when mixed. |
| 2 | TWEEN 20 | Cloudy after 3 days. |
| 3 | INCRONAM 30 | Slightly cloudy and yellow after 7 days. |
| 4 | TWEEN 80 | Orange precipitate formed in 7 days. |
| 5 | TRITON X-100 | Oily separation after 11 days. |
| 6 | RITATAINE | Globules formed after 25 days. |
| 7 | LONZAINE C | Yellowish after 59 days. |
| 8 | POLOXAMER 407 | Yellow precipitate formed at bottom of bottle |
| | | after 64 days. |
| 9 | LONZAINE CO | Clear but slightly yellow color after 64 days. |
| 10 | POLOXAMER 237 | Slightly cloudy after 64 days. |

1. TRITON X-200 is an anionic surfactant having the sodium salt of an alkylaryl polyether alcohol as the active ingredients, available from Union Carbide Corp. (Danbury, CT).
2. TWEEN 20 is a polyoxyethylene sorbitan monolaurate surfactant, also known as polysorbate 20, available from ICI Surfactants (Wilmington, DE).
3. INCRONAM 30 is a cocamidopropyl betaine amphoteric surfactant, available from Croda, Inc. (Edison, NJ).
4. TWEEN 80 is a polyoxyethylene sorbitan monolaurate surfactant, also known as polysorbate 80, available from ICI Surfactants (Wilmington, DE).
5. TRITON X-100 is a non-ionic surfactant, octylphenol ethoxylate, available from Union Carbide Corp. (Danbury, CT).
6. RITATAINE is a cocamidopropyl betaine amphoteric surfactant, available from Rita Corp. (Crystal Lake, IL).
7. LONZAINE C is a cocamidopropyl betaine amphoteric surfactant, available from Lonza, Inc. (Allendale, NJ).
8. POLOXAMER 407 is a polyoxypropylene-polyoxyethylene block copolymer non-ionic surfactant, available from BASF (Mount Olive, NJ).
9. LONZAINE CO is a cocamidopropyl betaine amphoteric surfactant, available from Lonza, Inc. (Allendale, NJ).
10. POLOXAMER 237 is a polyoxypropylene-polyoxyethylene block copolymer non-ionic surfactant, available from BASF (Mount Olive, NJ).

### Example 3

Measurement of Contact Angles of Liquid Carrier

In the following example, the wetting effect of solutions containing an acid component and different surfactants (surfactants) was evaluated. Solutions of 4.25% citric acid and 0.5% surfactant were prepared, unless otherwise indicated. The solutions were applied to bovine teeth and contact angle measurements were made. The results are reported in Table 2 below.

TABLE 2

| | | | Contact Angle |
|---|---|---|---|
| | | Immediate Contact | After One (1) |
| Sample | Surfactant | Angle | Minute |
| 1 | TWEEN 80 | 12° | 6° |
| 2 | LONZAINE CO | 15° | 6° |
| | *0.05% solution | | |
| 3 | LONZAINE CO | 9° | 5° |
| | *0.5% solution | | |
| 4 | POLOXAMER 237 | 19° | 11° |
| 5 | TWEEN 80 | 9° | 5° |

Measuring the contact angle provides a way of analyzing the wetting of a solid substrate by a liquid. The contact angle is related to the shape of the liquid resting on the solid substrate. A low contact angle means that the solution spreads or wets well. Solutions having relatively high contact angles do not wet as well as solutions having relatively low contact angles. As shown in Table 2 above, each of the surfactant solutions tested had a relatively low contact angle with the surface of the tooth. This means that the surfactants are effective in wetting out the surface tooth. It is expected that solutions prepared with these surfactants will be effective in breaking down the smear layer on the tooth's surface. An ASW 2500XE video contact angle measurement system available from AST Products (Billerica, Mass.) was used to measure the contact angle of the samples following the procedures recommended by the manufacturer.

Each of the patents, publications, and other documents mentioned or referred to in this specification is herein incorporated by reference in its entirety. Those skilled in the art will appreciate that numerous changes and modifications may be made to the preferred embodiments of the invention and that such changes and modifications may be made without departing from the spirit of the invention. It is therefore intended that the appended claims cover all such equivalent variations as fall within the true spirit and scope of the invention.

What is claimed is:

1. A method for removing smear layer from a prepared tooth surface comprising irrigating the surface with a sterile solution, comprising:
disinfectant;
surfactant selected from the group consisting of cocamidopropyl betaine compounds and copolymers of polyoxypropylene-polyoxyethylene; and at least 0.5% by weight of organic acid, said solution having a pH value of no greater than 4.

2. The method of claim 1, wherein the disinfectant is tetracycline.

3. The method of claim 1, wherein the disinfectant is doxycycline.

4. The method of claim 1, wherein the surfactant is a cocamidopropyl betaine compound.

5. The method of claim 1, wherein the surfactant is a polyoxypropylene-polyoxyethylene copolymer.

6. The method of claim 1, wherein the organic acid is citric acid.

7. The method of claim 1, wherein the organic acid has a pKa between 1.5 and 5.

8. The method of claim 1, wherein the pH value of the solution is between 1 and 4.

9. The method of claim 1, wherein the disinfectant is present in an amount of from 1 to 5 percent by weight of the solution.

10. The method of claim 1, wherein the surfactant is present in an amount of from about 0.05 to 1.5 percent by weight of the solution.

11. The method of claim 1, wherein the acid is present in an amount of from about 0.5 to 10 percent by weight of the solution.

12. The method of claim 11, wherein the acid is present in an amount of from about 3 to 6 percent by weight of the solution.

13. The method of claim 1, wherein the solution comprises 3% doxycycline, 0.5% coamidopropyl betaine compound, and 4.25% citric acid by weight.

14. The method of claim 1, wherein the solution comprises 3% doxycycline, 0.5% polyoxypropylene-polyoxyethylene copolymer, and 4.25% citric acid by weight.

15. A method for removing smear layer from a prepared orthopedic surface comprising irrigating the surface with a sterile solution, comprising:
disinfectant;
surfactant selected from the group consisting of cocamidopropyl betaine compounds and copolymers of polyoxypropylene-polyoxyethylene; and
at least 0.5% by weight of organic acid, said solutions having a pH value of no greater than 4.
